(19) United States
(12) Patent Application Publication
(10) Pub. No.: US 2023/0135711 A1
(43) Pub. Date: May 4, 2023

(54) ARTICLE FOR INFECTION PREVENTION FOR FOMITE MATERIALS

(71) Applicants: Daniel Lawrence Nahui Ollin CANESTARO-GARCIA, Medford, MA (US); Patrick Burns APFELD, Milwaukee, WI (US); Vatsal JAIPURIA, Jaipur, Rajsthan (IN); Ronald Skye LANKONE, Quincy, MA (US); Folia Water, Inc., Woburn, MA (US)

(72) Inventors: Theresa DANKOVICH, Medford, MA (US); Jonathan LEVINE, Medford, MA (US); Daniel Lawrence Nahui Ollin CANESTARO-GARCIA, Medford, MA (US); Patrick Burns APFELD, Milwaukee, WI (US); Vatsal JAIPURIA, Jaipur, Rajsthan (IN); Ronald Skye LANKONE, Quincy, MA (US)

(73) Assignee: Folia Water, Inc., Woburn, MA (US)

(21) Appl. No.: 17/917,797

(22) PCT Filed: Apr. 9, 2021
(86) PCT No.: PCT/US2021/026667

§ 371 Date: Oct. 7, 2022

Related U.S. Application Data
(60) Provisional application No. 63/007,421, filed on Apr. 9, 2020.

(51) Int. Cl. A41D 13/11; A61F 13/12; A61M 16/06; A61F 13/00

(52) U.S. Cl. CPC A41D 13/1192; A61F 13/122; A61M 16/06; A61F 13/00008; A61F 13/00063

(57) ABSTRACT

An embodiment is an article configured to inhibit or prevent pathogen growth. The article includes an inner layer configured to face a wearer's skin when the face covering article is applied to the face of the wearer; a middle layer adjacent to the inner layer; and an outer protection layer adjacent to the middle layer and opposite the inner layer and comprising a substrate and metal particles in the substrate, wherein the metal particles are configured to inhibit or prevent pathogen growth.

### TECHNICAL FIELD

The present disclosure relates to articles for inhibiting infection via fomite materials, and in particular to any cellulosic substrate, textile, polymeric articles for inhibiting infection.

### BACKGROUND

Antimicrobial and antiviral products are needed to address the health and safety of persons that are in contact with or visit clinical settings, such as medical offices, hospitals, etc. Apart from medical facilities, there is a need to incorporate antimicrobial and antiviral efficacy into products that commonly come into direct contact with the public, including fomite materials and products, such as paper-based products, textiles, nonwovens, etc., which are likely to carry infectious microorganisms. Preventing the spread of pathogens through various surfaces that come in contact with a person through routine use is an important public health issue.

Current prevention processes use antimicrobial paper-based filters impregnated with copper particles, as described in U.S. Pat. No. 9,611,153, as well as antimicrobial substrates that include silver particles, as described in International Patent Publication No. WO 2017/124057. There is a need, however, for enhanced antiviral and/or antimicrobial paper or textile substrate for use throughout medical facilities including hospital operating rooms and patient care areas, as well as essential services industries, such as grocery stores, trash collection, and food delivery.

### SUMMARY

An embodiment of the present disclosure is a face covering article. The face covering article includes an inner layer configured for placement adjacent to a wearer's skin when the face covering article is applied to the face of the wearer. The inner layer includes thermoplastic fibers and an outer region that defines a perimeter of the inner layer. The face covering article also includes a middle layer adjacent to the inner layer. The middle layer includes thermoplastic fibers and an outer region that defines a perimeter of the middle layer. The face covering article also includes an outer protection layer adjacent to the middle layer and opposite the inner layer. The outer protection layer has a blend of cellulosic fibers and thermoplastic fibers, and metal particles included therein. The metal particles being configured to inhibit or prevent pathogen growth. The layers include an outer region that defines a perimeter of the outer protection layer. At least portion of the outer region of the inner layer and the outer region of the outer protection layer are bonded together. The face covering article includes an attachment member configured to attach the face covering article to the wearer.

An embodiment of the present disclosure includes a disposable medical article. The disposable medical article also includes at least one substrate layer. The article also includes a protection layer having a substrate and metal particles in the substrate, the metal particles having a size that ranges from 1 to about 200 nanometers in at least one dimension, where the metal particles are configured to inhibit or prevent pathogen growth.

Another embodiment includes a wound dressing article. The wound dressing article also includes at least one substrate layer. The article also includes a protection layer having a substrate and metal particles in the substrate, the metal particles having a size that ranges from 1 to about 200 nanometers in at least one dimension, where the metal particles are configured to inhibit or prevent pathogen growth.

Another embodiment includes a packaging article. The packaging article also includes a protection layer having a substrate and metal particles in the substrate, the metal particles having a size that ranges from 1 to about 200 nanometers in at least one dimension, where the metal particles are configured to inhibit or prevent pathogen growth. Other embodiments of this aspect include corresponding computer systems, apparatus, and computer programs recorded on one or more computer storage devices, each configured to perform the actions of the methods.

Another embodiment includes an adhesive article. The adhesive article also includes a protection layer having a substrate and metal particles in the substrate, the metal particles having a size that ranges from 1 to about 200 nanometers in at least one dimension, where the metal particles are configured to inhibit or prevent pathogen growth. The article also includes an adhesive disposed along one side of the protection layer. The article also includes an optional cover layer that is directly adjacent to and faces the adhesive, the optional cover layer configured to be removed so as to expose the adhesive for place on a surface.

### BRIEF DESCRIPTION OF THE DRAWINGS

FIG. 1A illustrates an embodiment of substrates used in an article described herein;

FIG. 1B illustrates an embodiment of substrates used in the article described herein;

FIG. 1C illustrates an embodiment of substrates used in the article described herein;

FIG. 2A is a front view of a face covering article according to an embodiment of the present disclosure;

FIG. 2B is a schematic sectional side view of the face covering article shown in FIG. 2A;

FIG. 3A is a schematic front view of the inner layer of the article shown in FIGS. 2A and 2B;

FIG. 3B is a schematic front view of the middle layer of the article shown in FIGS. 2A and 2B;

FIG. 3C is a schematic front view of the outer protection layer of the article FIGS. 2A and 2B;

FIG. 4 is a partial exploded assembly view of the article shown in FIGS. 2A and 2B;

FIG. 5 is a detailed view of a portion of the face covering article shown in FIG. 2A;

FIG. 6 is a graph depicting results of virology testing over time for an outer protection layer sample with copper ion treatment;

FIG. 7 is a graph depicting results of virology testing over time for an outer protection layer sample with copper particle treatment;

FIG. 8 is a graph depicting results of virology testing over time for an outer protection layer sample with copper particle treatment;

FIG. 9 is a graph depicting results of virology testing over time for an outer protection layer sample with copper particle treatment;

FIG. 10 is a graph depicting results of virology testing over time for an outer protection layer sample with copper particle treatment; and

FIG. 11 is a graph depicting results of virology testing over time for an outer protection layer sample with silver treatment.

### DETAILED DESCRIPTION OF ILLUSTRATIVE EMBODIMENTS

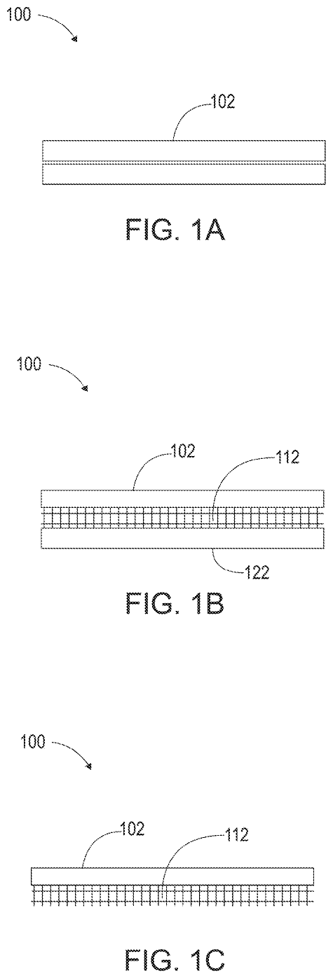

Embodiments of the present disclosure includes articles an article having metal particles deposited thereon that are suitable to inhibit or prevent infection from or by fomite surfaces or other high-touch surfaces. Referring to FIG. 1A-1C, the article 100 as described herein may comprise one or more various types of substrates 104 for various applications. FIG. 1A illustrates an exemplary multi-ply paper based antimicrobial materials for medical applications including medical tissue, towel, and exam table purposes. FIG. 1B illustrates a 3-ply material consisting of an outer protection layer 102 that includes metal particles, an inner layer 122 that may or may not include metal particles, and a middle layer 112 that may include a polypropylene. The 3-ply material illustrated in FIG. 1B may be used for disposable apparel purposes or as a face mask article as described further below. FIG. 1C illustrates a two-ply material with a metal nanoparticle protection layer 102 and a base layer 112 for sheets, placemats, curtains, and other non-apparel uses.

The one or more substrates 104 are formed with in situ reduction of metal salts in a continuous process resulting in formation of metal particles 108 on the surface of substrate components. The metal particles 108 may be nano-sized and micro-sized particles. For example, the metal particles 108 may be formed directly on the surface of fibers that form the substrates 104, as discussed further below. The metal particles 108 may include, for example, either silver or copper particles deposited or formed on the article itself. The article 100 described herein may be antimicrobial paper, antiviral paper, textile articles, nonwoven articles, or combinations thereof, configured to combat, inhibit, and/or prevent the spread of pathogenic microorganisms through fomite surfaces such as clothing, furniture, personal protective equipment (PPE), packaging, etc. The article 100 may have antibacterial, anti-fungal, antiviral, and anti-yeast properties.

Typical substrates require modification to create an efficacious infection control article that will limit the persistence of microorganisms on fomite surfaces. To produce an antimicrobial material to prevent infection from touching a fomite surface, the material must absorb microbial contaminated aerosols and inactivate the microbes within several minutes to prevent transmission of infectious pathogens to other people. There is an advantage for using hydrophilic material, such as paper or cellulosic polymer textiles, because the material is naturally very absorbent of aerosols (water-based droplets containing microorganisms). By adding antimicrobial metal particles, such as nano-silver and nano- and micro-copper particles, to the surface of fibers, any droplets that contact the surface of the substrate will be quickly absorbed into the fibrous substrate and brought in direct contact with the metallic particles. The direct contact will result in a rapid disinfection process. In some examples, the disinfection process may last a few minutes. To achieve antimicrobial or anti-viral elimination in the short-required time span, the recommended range for such metallic nanoparticle precursors such as silver nitrate or other aqueous silver salt and/or the aqueous copper salt should be between 1 ppm and 10,000 ppm.

Cellulosic materials that can be used include the following: wood pulp in wet laid paper-making, air laid fluff pulp, cotton, viscose, rayon, and cellulosic blends including materials comprised of polypropylene cellulose, polyethylene-terephthalate cellulose, and other cellulosic synthetic polymer blends. The particular cellulosic material that would be utilized is dependent upon the particular application. The process for adding metal nanoparticles to the various cellulosic materials will depend upon the processing for each type of cellulose.

The substrate 104 can be formed from a wide range of materials, including fibers. The substrate 104 may include paper substrates, paper laminates, nonwovens, nonwoven laminates, textiles, textile laminate, or laminates of a paper, nonwovens, and/or textiles. The substrate 104 may include any range of component types, such as cellulosic fibers or polymeric fibers as needed. For example, fibers may include, but are not limited to natural cellulosic fibers, such as natural wood fibers, natural cotton fibers, synthetic cellulosic fibers, blends thereof, and other hydrophilic fibrous fibers.

The article 100 described herein can be made by process and materials as described in International Patent Publication No. WO2017124057, the entire disclosure of which is incorporated by reference herein. For example, a base substrate may have the in-situ synthesis of the metallic nanoparticles occur either in-line on a paper machine or off-line on a coating machine. By extension, the metallic ions may be absorbed to the substrate components, such as in the cellulosic fibers prior to substrate formation through pulp treatment metal ion impregnation processes and reduced to metallic nanoparticles during the paper making or coating processes.

In order to increase wettability of hydrophobic fibrous nonwoven materials (wet-laid or air-laid products), corona discharge treatment (“CDT”) may be utilized to increase uptake of the coating solution. A variety of nonwovens (plastic and cellulose based) may therefore be coated because of use of CDT. In addition, for air-laid products, to create the metal particles 108 in the substrate 104, metal particle precursor chemicals may be added to fibers along with application of a binder (e.g. with the latex binder typical in air-laid processes). Such binders are typically added in a spray or foam to the fiber network during the air-laid process, then activated by heat from the dryers to set the binder. Because the metal particle synthesis is also catalyzed by thermal energy in a continuous process, the spray or foam application of a mixture of binder, metal salts, and other reducing agents would be added to the substrate. This would result in metal nanoparticle formation during the air-laid paper-making process. The result may provide antimicrobial and/or antiviral articles.

The continuous processes as described herein have number of advantages over the batch processes. For instance, in contrast to the batch methods, the continuous processes as described herein allows large quantities of nanoparticle embedded paper to be produced in a matter of minutes. The continuous processes as described herein may utilize, for example, Dixon Coater, a Fourdrinier pilot machine and other commercial large-scale paper forming lines. A comparatively small Dixon coater, for example, coats and dries at 12 inch roll of paper at 280 linear feet of paper per minute (ft/min) running at full speed. A Fourdrinier pilot machine can produce speeds, which typically range from 10 ft/min to 300 ft/min, that far outstrip the levels of production possible in previously disclosed inventions (Dankovich, 2015). Larger, commercial paper forming lines are capable of even higher throughput, typically in the range of 500 ft/min to 2500 ft/min. The previously disclosed conventional batch methods for nanoparticle synthesis have not been readily adapted to this powerful technology, and thus they have not been widely adopted.

Rather than synthesizing metal nanoparticles in individual filter sheets, as previously described (Dankovich, 2014) in batch methods, the present disclosure includes a continuous manufacture of bulk quantities of metal nanoparticles directly on the substrate. Thus, embodiments described herein include methods for synthesizing metal nanoparticles within substrates in seconds rather than minutes or hours as compared to batch methods. The result is a significant increase in production speeds that yield metal nanoparticles embedded cellulosic materials. Furthermore, the inventive concepts described herein do not significantly alter the surface chemistry of pulp or paper during the wet formation of the substrate. Furthermore, the processes as described herein do not significantly alter the physical properties of the resultant substrate as compared to the same process without the nanoparticle synthesis step. The in situ method to form nanoparticles directly on the fiber surfaces has a few other advantages over previous batch methods. For example, the overall levels of metal nanoparticles that can be formed and retained in the substrate is much higher with an in situ synthesis process as described herein, at least compared to the absorption process of the nanoparticles (Dankovich and Gray, 2011). In situ synthesis methods as described herein can prevent of the excessive losses of expensive metal reagents during manufacturing processes and product usage phases. There may be little to no loss of metal precursors in this manufacturing process due to the re-circulation of the solution in the application unit.

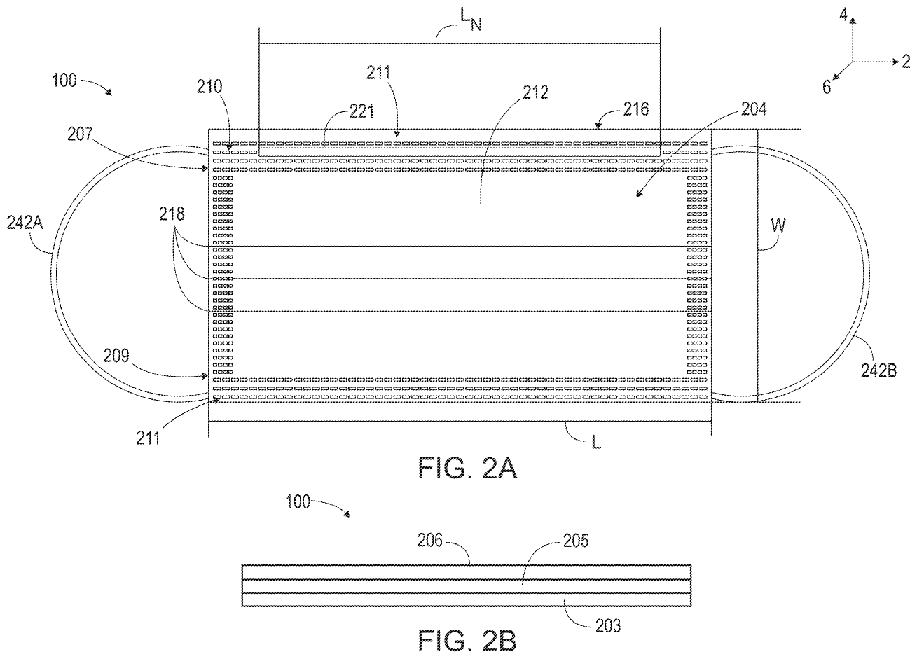

In the illustrated embodiment (FIGS. 2A-5), the present disclosure includes a face mask application of the substrate 104 with metal particles 108 bonded to the fibers thereof. In the present disclosure, 104 and 204 may be used interchangeably to identify the substrate or substrate layer; and 108 and 208 may be used interchangeably to identify the metal particles.

Referring to FIGS. 2A and 2B, the article 100 is an antiviral and anti-microbial face covering article configured to cover the mouth and nose of a user. The face covering article 100 may be a medical or surgical face mask. In the illustrated embodiment, the face covering article is a 3-ply material although more than three plies may be used.

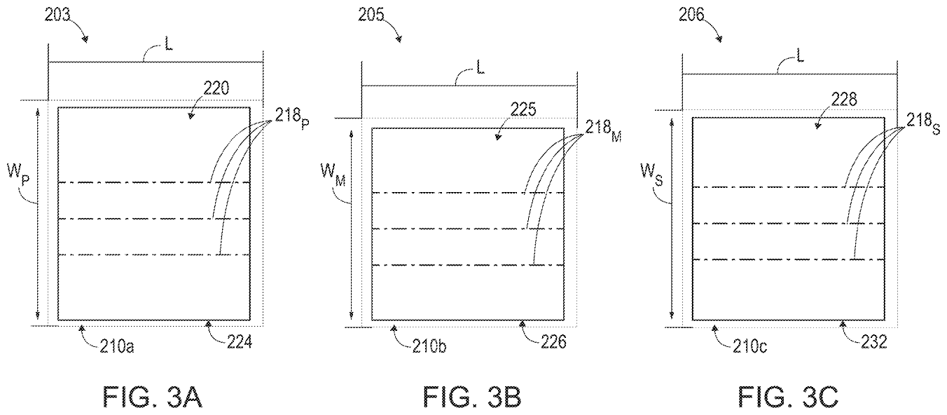

As illustrated in FIGS. 2A-4, the face covering article 100 includes an inner layer 203, a middle layer 205, and an outer protection layer 206 adjacent to the middle layer 205. The inner layer 203 is configured to face the user's skin when the face covering article 100 is applied to the face of the user. The outer protection layer 206 is opposite the inner layer 203 and configured to face away from the wearers skin when the face covering article 100 is applied to the face of the user. Each layer 203, 205, and 206 further includes an outer region 210a, 201b, 210c (FIGS. 3A-3C) defining an outer perimeter 211 of the article 100. The outer perimeter 211 extends around at least a mouth and nose of the user when the face covering article 100 is worn by the user. In the illustrated embodiment, the inner layer 203, the middle layer 205, and the outer protection layer 206 are configured to be bonded together, as explained further below.

The face covering article 100 has a non-expanded state and an expanded state. In the non-expanded state, the face covering article 100 has a length L that extends along a longitudinal direction 2 and a width W₁ that extends along a vertical direction 4 that is opposite the longitudinal direction 2. In the illustrated embodiment, the length L may be about 175.0 cm, and the width W may be about 95.0 mm. In alternate embodiments, the dimensions of the article 100 may vary.

The article 100 has an upper portion 207 and a lower portion 209 opposed to the upper portion 207 along the vertical direction 4. Further, the article 100 has a face portion 212 and a back portion 216 opposed to the face portion 212 along a lateral direction 6 that is perpendicular to the longitudinal direction 2 and vertical direction 4. The face portion 212 is configured to be exposed to the user's surroundings and the back portion 216 is configured to face the user's skin when the face covering article is applied to the face of the user. The face covering article 100 includes one or more pleats 218 extending along the length L on the face portion 212 of the article 100. The pleats 218 are configured to expand the face covering article 100 when the article 100 is applied to the user's face and worn. The pleats 218 are further configured to enable the article 100 to be threefold.

The article 100 further includes a nose piece or nose bridge piece 221. The nose piece 221 is configured to conform a fit of the article 100 to a bridge of the user's nose when the article 100 is applied to the user's face and worn. The nose piece 221 may be made of aluminum or a bendable plastic. The nose piece 221 may be positioned on the upper portion 207 of the article and may be welded in place. In the illustrated embodiment, the nose piece may have a length LN of about 100.0 mm and a width of about 3.0 mm and a thickness range of about 0.3 to 0.8 mm. In an alternative embodiment, the nosepiece may vary.

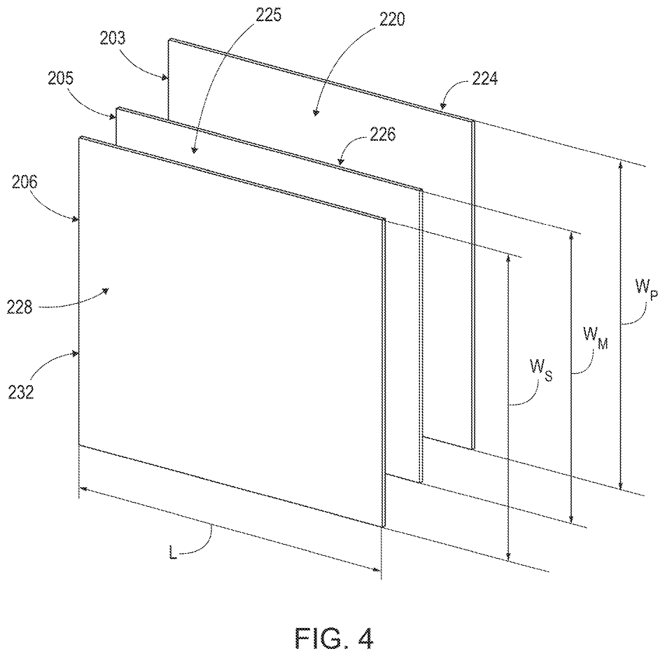

Referring to FIGS. 3A-4, the inner layer 203 includes an upper surface 220 and a lower surface 224 opposite the upper surface. The lower surface 224 is adjacent to a user's skin when the face covering article 100 is applied to the face of the user.

The inner layer 203 includes one or more pleats 218_{S} that extend parallel to each other along the length L of the inner layer 203. The one or more pleats 218_{S} are configured to deform the inner layer 203 between an expanded state and a non-expanded state when the face covering article 100 is applied to the user's face and worn. In the expanded state, the inner layer 203 has a length L of about 175.0 mm and a thickness T of about 0.16 mm. The inner layer 203 has a width WP that is about 195.0 mm. In an alternative embodiment, the length L, the thickness T, and the width WP of the inner layer 203 may vary.

The inner layer 203 may be formed from nonwoven laminate materials. For example, the inner layer 203 may be a spunbond substrate, a meltblown substrate, or laminates of spunbond and meltblown substrates. In such embodiment, inner layer 203 may be laminates of SMS, SMMS, etc. In the illustrated embodiment, the inner layer 203 are formed from polypropylene (PP) laminates as described above. Furthermore, the inner layer 203 may have a basis weight that ranges from 20.0 gsm (grams per square meter) to about 80.0 gsm.

The middle layer 205 includes one or more pleats 218_{M} that extend parallel to each other along the length L of the middle layer 205. The one or more pleats 218_{M} are configured to deform the middle layer 205 between a non-expanded state and an expanded state when the face covering article 100 is applied to the user's face and worn. In the expanded state, the middle layer 205 has a length L of about 175.0 mm and a thickness T of about 0.16 mm. The middle layer 205 has a width W_{M} that is smaller than the than the width WP of the inner layer 203. In the illustrated embodiment, the width W_{M} is about 175.0 mm. In an alternative embodiment, the length L, the thickness T, and the width W_{M} of the middle layer 205 may vary.

The middle layer 205 includes a front surface 225 and a back surface 226 opposite the front surface 225. The back surface 226 is adjacent to the upper surface 220 of the inner layer 203 such that the middle layer 205 lays atop the inner layer 203 and has the same dimensions as the inner layer 203. The middle layer 205 may be formed from nonwoven laminate materials. For example, the middle layer 205 may be a spunbond substrate, a meltblown substrate, or laminates of spunbond and meltblown substrates. In such embodiment, middle layer 205 may be laminates of SMS, SMMS, etc. In one example, the middle layer is a meltblown electrostatically charged substrate. In the illustrated embodiment, the middle layer 205 are formed from polypropylene (PP) laminates as described above.

The outer protection layer 206 is configured to face outwardly away from the wearers face when worn. Furthermore, the outer protection layer 206 is configured to inhibit or prevent antimicrobial growth and viral spread. As illustrated, the outer protection layer includes a top surface 228 and a bottom surface 232 opposite the top surface 228. The bottom surface 232 is adjacent to the front surface 225 of the middle layer 205 such that the outer protection layer 206 lays atop the middle layer 2045 and has the same dimensions as the middle layer 205 and the inner layer 203.

The outer protection layer 206 also includes one or more pleats 218p that extend parallel to each other along the length L of the outer protection layer 206. The one or more pleats 218_{P} are configured to deform the outer protection layer 206 between a non-expanded state and an expanded state when the face covering article 100 is applied to the user's face and worn. In the expanded state, the outer protection layer 206 has a length L of about 175.0 mm and a thickness T of about 0.16 mm. The outer protection layer 206 has a width WS that is smaller than the than the width WP of the inner layer 203 and larger than the width W_{M} of the middle layer 205. In the illustrated embodiment, the width WS is about 183.0 mm. In an alternative embodiment, the length L, the thickness T, and the width WS of the outer protection layer 206 may vary.

The outer protection layer 206 is comprised of a substrate with metal particles formed at least on the surface thereof. In the example shown, the outer protection layer is a nonwoven material that includes a blend of staple cellulosic fibers and staple thermoplastic fibers. In one specific example, the outer protection layer is a nonwoven material that includes a blend of staple cellulosic fibers and staple polypropylene fibers (e.g. PP fibers). The outer protection layer 206 may have between 50% and 90% by weight of cellulosic fibers and between about 10% and 50% by weight of thermoplastic fibers. The outer protection layer 206 may have a basis weight between about 16.0 gsm and about 45.0 gsm. In one example, the outer protection layer has a basis weight of at least 16.0 gsm. In another example, the outer protection layer has a basis weight up to about 45.0 gsm. In another example, the outer protection layer has a basis weight between about 20.0 gsm and 40.0 gsm. In another example, the basis weight for the outer protection layer 206 is about 24.0 gsm.

The outer protection layer 206 may include a plurality of metal particles 208. The metal particles 208 are configured to inhibit or prevent pathogen growth. The metal particles 208 have a size that ranges from 1 nanometer to about 200 nanometers in at least one dimension. In the illustrated embodiment, the metal particles 208 include at least one of: silver, gold, platinum, palladium, aluminum, iron, zinc, copper, cobalt, nickel, manganese, molybdenum, cadmium, iridium, and a mixture thereof. In an embodiment, the metal particles are formed in situ, during substrate processing so as to establish between 0.05 gsm to about 1.5 gsm of metal particles on the outer protection layer 206. In one example, the outer protection layer 206 includes at least 0.05 gsm of metal particles. In another example, the outer protection layer 206 includes up to about 1.5 gsm of metal particles. In another example, the outer protection layer 206 includes about 0.6 gsm of metal particles. In another example, the outer protection layer 206 may have between 0.5% and 5.0% by weight of silver per gram of the substrate of the outer protection layer 206. In another example, the outer protection layer 206 may have a silver loading of about 2.2% by weight of silver per gram of the substrate of the outer protection layer 206. The metal particles may have a size that ranges from 1 to about 200 nanometers in at least one dimension. The result, as explained further blow, is a face covering articles with an antiviral log reduction of at least 4.0, an average filtration efficiency of at least 99.71%, and a bacterial filtration efficiency of at least 99.37%.

In one embodiment, the outer protection layer 206 may be made with 99.9% pure silver. In one example, the outer protection layer 206 includes an antimicrobial silver preservative coating that releases silver into microbe-laden water droplets on the protection layer 206. The outer protection layer 206 may have between 0.2% and 1.0% by weight of silver per gram of the substrate of the outer protection layer 206. In another example, the outer protection layer 206 may have a silver loading of about 0.5% by weight of silver per gram of the substrate of the outer protection layer 206. This configuration inhibits microbe colonization on both the outer protection layer 206 and the article 100. The outer protection layer 206 may be subjected to ultraviolet treatment. Ultraviolet treatment may be used to drive silver fixation towards completion. This configuration may enhance resistance of the outer protection layer 206 to environmental factors such as aging or silver release. In one example, the outer protection layer 206 may be subjected to ultraviolet treatment and high ultraviolet exposure such that 1) silver fixation is greater than 90% fixation on the substrate and 2) the outer protection layer 206 exhibits a greater resistance to aging (i.e. no change in appearance or darkening of the outer protection layer 206).

The outer protection layer 206 is made of a blend suitable for ultrasonic welding with the middle layer 205 and the inner layer 203. This configuration enhances the breathability and pressure-drop performance of the article 100. As shown, the inner layer 203, the middle layer 205, and the outer protection layer 206 are bonded together at their respective outer regions via ultrasonic welding. In an alternative embodiment, however, the inner layer 203, the middle layer 205, and the outer protection layer 206 may be held together by other thermal or chemical means, such as heat or embossing. In alternative embodiments, the inner layer 203, the middle layer 205, and the outer protection layer 206 may further be held together by other attachment means.

In this configuration, the outer protection layer 206, and thus the face covering article 100, may be antimicrobial and/or antiviral through the addition of metal particles, such as silver or copper. In another embodiment, the metallic cellulosic component may be disposed just inside the outer protection layer 206, to create a 4-ply face covering article. In an alternative embodiment, the metallic cellulosic material can be added as a protective patch atop the outer protection layer 206.

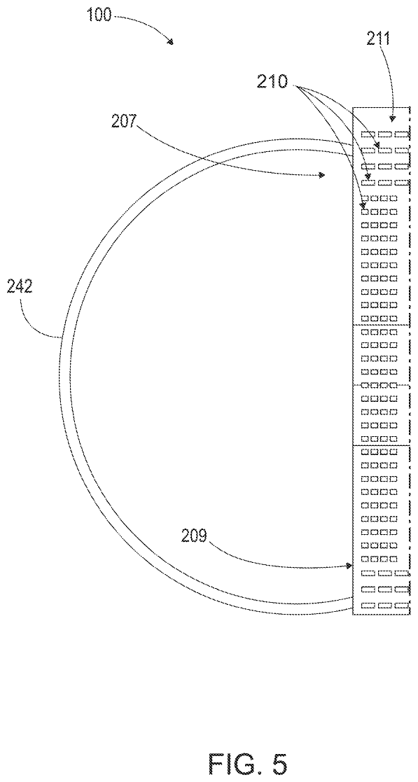

Referring to FIG. 5, the face covering article 100 further includes an attachment member 240 configured to attach the face covering article 100 to the user. In the illustrated embodiment, the attachment member 240 includes one or more bands 242 coupled to the upper portion 207 and the lower portion 209 of the face covering article 100. In the illustrated embodiment, the attachment member 240 includes a first band 242A coupled to the upper portion 207 and the lower portion 209 at one end of the face covering article 100, and a second band 242B coupled to the upper portion 207 and the lower portion 209 at the opposing end of the face covering article 100. The first band 242A and the second band 242B may be coupled at the outer perimeter 211 of both the inner layer 203 and the outer protection layer 206. In the illustrated embodiment, the first band 242A and the second band 242B are elastic bands having a length of about 175.0 mm and a width of about 3.0 mm. The bands 242A, 242B are coupled to the outer perimeter 211 via welds. In this configuration, the bands 242A, 242B may be stretchable and may be easily applied to the user's face proportions. In alternative embodiments, other materials and attachment means may be utilized in the attachment member 240.

### EXAMPLES

### Example 1: Preparation of Metal Particles Added to Protection Layer and Testing of Properties

In this example, virology testing on paper material samples impregnated with silver and/or copper substances was used to test the antiviral efficacy of the samples. The model system used was bacteriophage (as the viral proxy) and Escherichia coli (as the host proxy). The plaque assay was used as the efficacy measurement method. The plaque assay is used for virus isolation/purification and to determine viral titers. A viral titer, a.k.a. viral load or burden, is a numerical expression quantifying virus in a given volume of fluid. The plaque assay is an optimized virological method developed to count and measure infectivity of bacteriophages.

Materials and Methods

Seventeen (17) base papers were provided as samples. Samples 1-4, 9-13, and 16-17 were a polypropylene-cellulose blend. These base papers composed about 25-35% polypropylene and about 65%-75% cellulose. Samples 5-8 and 14-15 were a pure 100% cellulose blend. Some of the base papers were impregnated with either silver or copper substances; others were not. The identity of each paper was blinded to the principal investigator, i.e. papers were labeled with numbers ranging 1 to 17 with no other distinguishing marks. Each of the papers were cut and fitted into a 24-well plate. For each cut paper, three (3) replicates were tested efficacy over 4 different incubation timepoints, i.e. 5 minutes, 30 minutes, 1 hour, and 4 hours. The experimental set-up is shown in the images to the right. The efficacy of the papers' surface to mitigate viral transmission and replication was performed using the method reported in Doremalen, et al. (2020).

Bacteriophage was used in this comparative study to demonstrate protocol verification. P1 phage, a temperate bacteriophage that infects Escherichia coli (E. coli) and some other bacteria, was used as a proxy virus.

The experimental design was set-up in seven (7) separate but interconnected tasks:

1. Paper material preparation

2. Phage propagation

3. Viral harvest

4. Agar plate preparation

5. Bacterial culture

6. Bacterial cell culture exposure to viral harvest

7. Phage titer determination

A known amount of virus titer stock solution was deposited on the surfaces of each cut paper material. After a 5-minute incubation time at room temperature (21-23° C.) and 40% relative humidity, the first set of materials were analyzed for virus replication. One (1) milliliter of collection medium was used to recover virus. The virus titer collection medium was added to the E. coli culture, left in 37° C. incubator for 24 hours, and subsequently quantified by counted plaque forming units (PFUs). The titer of a virus stock was calculated in PFU per milliliter (mL) (Dulbecco & Vogt. 1953).

After each petri dish was counted for plaques, the values were recorded plotted as PFUs over incubation time. using plates with between 5-100 plaques, the Log Reduction Value was calculated using the following equation:

$$LogReductionValue = {log}_{10} ( \frac{StartingViralLoad}{EndingViralLoad} )$$

Where Starting Viral Load was 1.45×10⁶ PFU/mL for Set A and B Papers and 1.75×10⁷ PFU/mL. for Set C Papers. Ending Viral Load was the PFU value assessed after each incubation time period (e.g. 100 counted plaques is equivalent to 100 PFU/mL). Each Log Reduction Value is included in the results section table immediately to the right of each Plaque Count. Starting Viral Load was calculated as the average PFU/mL from the titer curve (Sanders 2012; Andersson and Lood 2019; Bear 2014; Mendoza 2020; LaBarre 2001).

Results

Results of this assay show papers loaded with either silver or copper substances exhibited antiviral properties relative to control papers (i.e. papers not loaded with silver or copper substances). The papers exhibiting the highest amount of antiviral efficacy were Sample Numbers 12 and 16. The values of each sample's log reduction value are included in Table 1 and Table 2 below.

TABLE 1

| | | | | Log | Log |
|---|---|---|---|---|---|
| | | | | Reduction | Reduction |
| Sample | Sample | | | Value @ | Value @ |
| # | Grade | Silver | Copper | 30 min | 1 hr |
| 1 | Dynapore | Uncoated | Uncoated | NA | NA |
| 2 | Dynapore | Low | | 4.77 | 4.77 |
| 3 | Dynapore | Med | | 4.77 | 4.77 |
| 4 | Dynapore | High | | 4.77 | 5.04 |
| 5 | Coffee filter | Uncoated | Uncoated | NA | NA |
| 6 | Coffee filter | Low-Med | | 4.77 | 4.95 |
| 7 | Masking tape | Uncoated | Uncoated | NA | NA |
| 8 | Masking tape | Low-Med | | 4.77 | 5.09 |
| 9 | Coffee filter | Uncoated | Uncoated | NA | NA |
| 10 | Coffee filter | Med | | 4.77 | 4.98 |
| 11 | Face mask | Uncoated | Uncoated | NA | NA |
| | P-P blend | | | | |
| 12 | Face mask | Low-Med | | 4.77 | 5.31 |
| | P-P blend | | | | |
| 13 | Dynapore | | Particles | 3.71 | 3.97 |
| 14 | Coffee filter | | Particles | 3.69 | 3.90 |
| 15 | Masking tape | | Particles | 3.85 | 4.02 |
| 16 | Coffee filter | | Particles | 3.87 | 5.05 |
| 17 | Coffee filter | | Ions | 3.84 | 4.41 |

TABLE 2
Log Reduction Values of Samples with Coat
Weight and % Silver and Copper Loading

| | | |
|---|---|---|
| Coat | Silver | Cu |

| | | | |
|---|---|---|---|
| weight | loading | loading | Log Reduction |

| # | (gsm) | (%) | (%) | 30 min | 1 HR |
|---|---|---|---|---|---|
| 1 | — | — | | NA | NA |
| 2 | 17.6 | 0.38% | — | 4.77 | 4.77 |
| 3 | 23.5 | 0.48% | — | 4.77 | 4.77 |
| 4 | 35.2 | 0.58% | — | 4.77 | 5.04 |
| 5 | — | — | — | NA | NA |
| 6 | 15.3 | 0.51% | — | 4.77 | 4.95 |
| 7 | — | — | — | NA | NA |
| 8 | 21.4 | 0.63% | — | 4.77 | 5.09 |
| 9 | | — | — | NA | NA |
| 10 | 30 | 0.53% | — | 4.77 | 4.98 |
| 11 | | — | — | NA | NA |
| 12 | | ? | — | 4.77 | 5.31 |
| 13 | 1 | — | 2.6% | 3.71 | 3.97 |
| 14 | 1 | — | 3.6% | 3.69 | 3.9 |
| 15 | 2.9 | — | 6.9% | 3.85 | 4.02 |
| 16 | 2.3 | — | 5.7% | 3.87 | 5.05 |
| 17 | 3 | — | 7.3% | 3.84 | 4.41 |

| Silver coating: | Copper coating: |
|---|---|
| silver nitrate | copper |
| glycerol dextrose | copper oxides |

The following tabulated data includes paper sample numbers 12, 13, 14, 15, 16, and 17.

TABLE 3
Average Plaque Forming Units Over Time For Samples 12-17

| | | | Sample | Sample | Sample | Sample | Sample | Sample |
|---|---|---|---|---|---|---|---|---|
| | | | No. 17 | No. 16 | No. 15 | No. 14 | No. 13 | No. 12 |
| | | | AVG | AVG | AVG | AVG | AVG | AVG. |
| PFUs | at 5 | min | 300 ± 0 | 300 ± 0 | 300 ± 0 | 300 ± 0 | 300 ± 0 | 300 ± 0 |
| | at 30 | min | 212 ± 36 | 196 ± 1 | 206 ± 14 | 299 ± 2 | 283 ± 28 | 300 ± 0 |
| | at 1 | hr | 56 ± 3 | 13 ± 6 | 138 ± 26 | 181 ± 4 | 157 ± 27 | 85 ± 18 |
| | at 4 | hr | 0 ± 0 | 0 ± 0 | 0 ± 0 | 0 ± 0 | 0 ± 0 | 0 ± 0 |

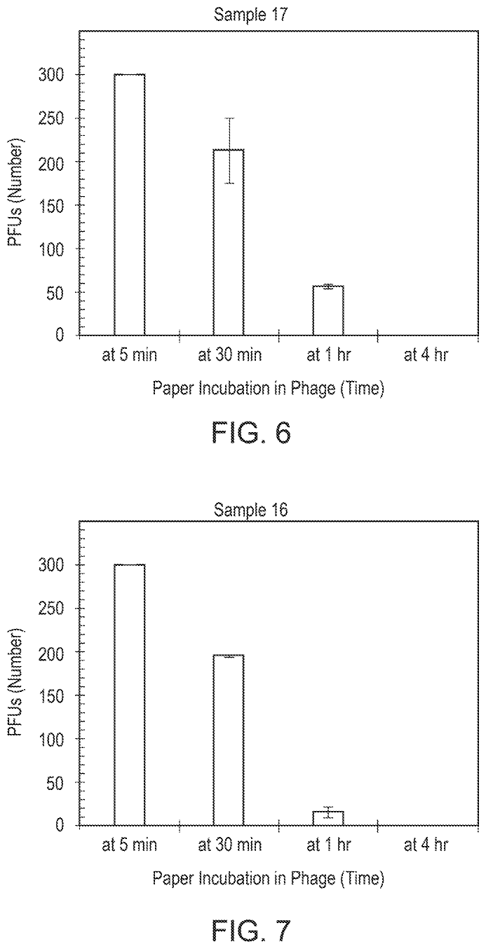

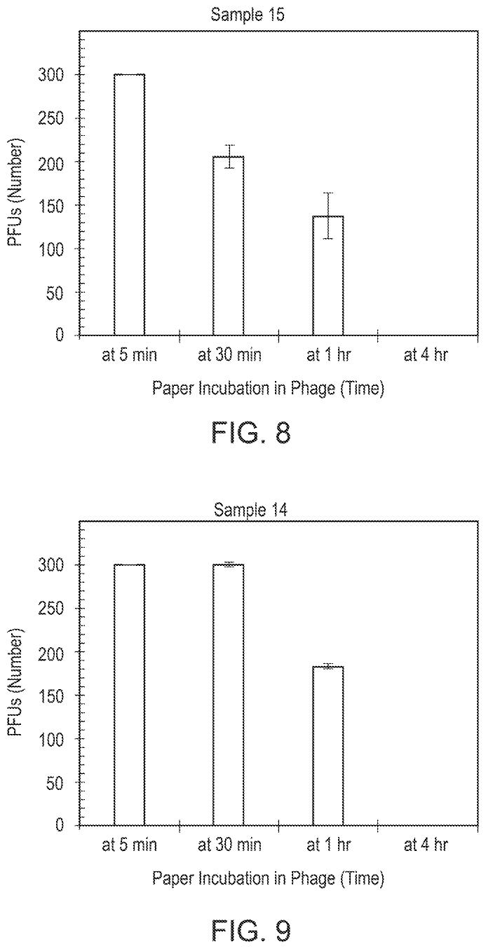

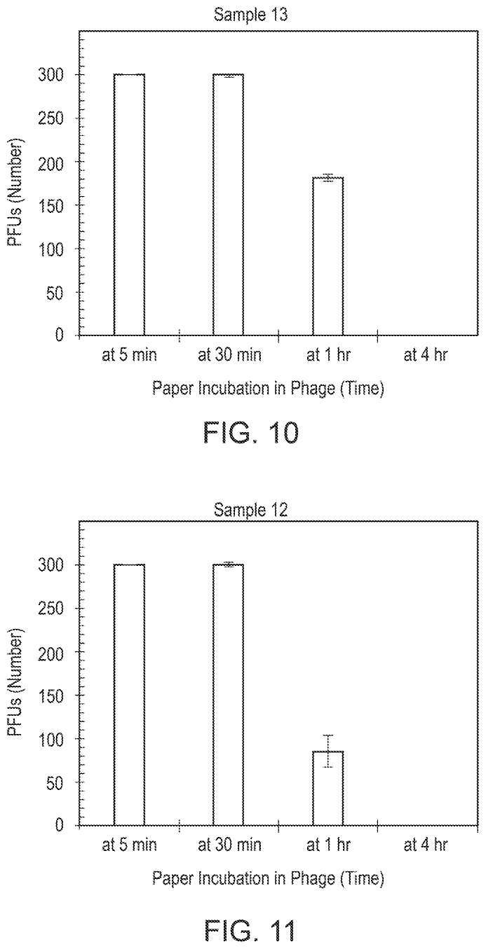

The results of the study with respect to average plaque forming units over time for Samples 12-17 are shown in FIGS. 6-11, respectively.

Paper materials in Set A (maroon; 13, 14, 15, 16, and 17) were coated with copper substances and performed very well in the antiviral efficacy testing. The plaque assay resulted in average PFU counts with minor standard deviations in the replicates. Preliminary evidence revealed that Sample Number 16 (Ahlstrom-Coffee filter-Copper Particles) performed the best as compared to all papers included in this set, i.e. the paper was effective at reducing plaque formation at the lowest incubation time period as well as the highest log reduction value.

None of the paper materials in Set B (white; 1, 5, 7, 9, and 11) performed well in the antiviral testing. Preliminary results show that no antiviral efficacy was seen in any papers tested.

Paper materials in Set C (brown; 2, 3, 4, 6, 8, 10, and 12) were coated with silver substances and most performed very well in the antiviral efficacy testing. Like the results in Set A, the plaque assay resulted in average PFU counts with minor standard deviations in the replicates. Preliminary evidence revealed that Sample Number 12 (Ahlstrom-Face mask P-P blend-Silver Low-Med) performed the best as compared to all papers included in this set, i.e. the paper was effectively reduced plaque formation at the highest log reduction value.

### Example 2: Particle Filtration Efficiency

In this example, a procedure was performed to evaluate the non-viable particle filtration efficiency (PFE) of the test article.

Materials and Methods

Monodispersed polystyrene latex spheres (PSL) were nebulized (atomized), dried, and passed through the test article. The particles that passed through the test article were enumerated using a laser particle counter.

A one-minute count was performed, with the test article in the system. A one-minute control count was performed, without a test article in the system, before and after each test article. Control counts were performed to determine the average number of particles delivered to the test article. The filtration efficiency was calculated using the number of particles penetrating the test article compared to the average of the control values. During testing and controls, the air flow rate is maintained at 1 cubic foot per minute (CFM)±5%.

The procedure employed the basic particle filtration method described in ASTM F2299, with some exceptions; notably the procedure incorporated a non-neutralized challenge. In real use, particles carry a charge, thus this challenge represents a more natural state. The non-neutralized aerosol is also specified in the FDA guidance document on surgical face masks. All test method acceptance criteria were met. Testing was performed in compliance with US FDA good manufacturing practice (GMP) regulations 21 C.F.R. Parts 210, 211 and 820.

Test Article: 102420A—7 samples; 102420B—7 samples; 102420E—7 samples

Test Side: Inside

Area Tested: 91.5 cm2

Particle Size: 0.1 μm

Laboratory Conditions: 16 Mar. 2021: 21.2° C., 22% relative humidity (RH) at 2204; 21.1° C., 22% RH at 2252; 20.9° C., 22% RH at 2308 21 Mar. 2021: 21.2° C., 22% RH at 1401; 21.0° C., 22% RH at 1446

Results

The results of the study are shown in Tables 4-6 below.

TABLE 4
Test Article: 102420A

| Test | Test | Average | Filtration |
|---|---|---|---|
| Article | Article | Control | Efficiency |
| Number | Counts | Counts | (%) |
| 1 | 43 | 13,392 | 99.68 |
| 2 | 34 | 13,340 | 99.75 |
| 3 | 38 | 13,131 | 99.71 |
| 4 | 38 | 12,764 | 99.70 |
| 5 | 38 | 12,922 | 99.71 |
| 6 | 32 | 12,504 | 99.74 |
| 7 | 34 | 11,547 | 99.71 |

The average filtration efficiency for this test article was 99.71%, with a standard deviation of 0.024.

TABLE 5
Test Article: 102420B

| Test | Test | Average | Filtration |
|---|---|---|---|
| Article | Article | Control | Efficiency |
| Number | Counts | Counts | (%) |
| 1 | 37 | 11,995 | 99.69 |
| 2 | 30 | 12,466 | 99.76 |
| 3 | 41 | 12,135 | 99.66 |
| 4 | 45 | 11,434 | 99.61 |
| 5 | 28 | 11,785 | 99.76 |
| 6 | 45 | 11,365 | 99.60 |
| 7 | 44 | 11,019 | 99.60 |

The average filtration efficiency for this test article was 99.67%, with a standard deviation of 0.071.

TABLE 6
Test Article 102420E

| Test | Test | Average | Filtration |
|---|---|---|---|
| Article | Article | Control | Efficiency |
| Number | Counts | Counts | (%) |
| 1 | 52 | 12,331 | 99.58 |
| 2 | 37 | 13,258 | 99.72 |
| 3 | 40 | 13,731 | 99.71 |
| 4 | 62 | 14,017 | 99.56 |
| 5 | 44 | 14,341 | 99.69 |
| 6 | 64 | 14,715 | 99.57 |
| 7 | 22 | 12,463 | 99.82 |

The average filtration efficiency for this test article was 99.66%, with a standard deviation of 0.100.

### Example 3: Bacterial Filtration Efficiency

In this example, a procedure was performed to evaluate the bacterial filtration efficiency (BFE) of the test article.

Materials and Methods

Five specimen test articles were utilized. The specimens were conditioned for 4-hours at 20.4-22.1° C. and 83-86% RH. Test set up involved the following:

| | |
|---|---|
| Area of Test Specimen (cm²) | 48.3 |
| Specimen Side Facing Challenge | Inside of Mask |
| Flow Rate (LPM) | 28.3 |
| Averaged + Control Plate Count | 2542 |
| Mean Particle Size (μm) | 3 |

Results

Results of the bacterial filtration efficiency testing were recorded as follows:

TABLE 7
Medical Face Mask Barrier Testing

| | Plate Count | | | | |
|---|---|---|---|---|---|
| | Mask Specimen | | | | |
| Stage | 1 | 2 | 3 | 4 | 5 |
| Stage 1 | 0 | 0 | 0 | 0 | 0 |
| Stage 2 | 0 | 0 | 0 | 0 | 0 |
| Stage 3 | 0 | 0 | 0 | 0 | 0 |
| Stage 4 | 0 | 2 | 1 | 8 | 0 |
| Stage 5 | 3 | 6 | 3 | 7 | 0 |
| Stage 6 | 1 | 2 | 1 | 1 | 0 |
| Plate Count Total | 4 | 10 | 5 | 16 | 0 |
| % BFE | 99.84 | 99.61 | 99.80 | 99.37 | >99.9 |

### Example 4: Microbial Cleanliness

In this example, microbial cleanliness (bioburden) of the test article were conducted on the test article.

Materials and Methods

A minimum of 5 specimen test articles were utilized. Testing was conducted using standard test protocol (STP) Number STP 0036 (Rev. 15). The testing was conducted in accordance with EN 14683:2019 and ANSI/AAMI/ISO 11737-1:2018. The counts determined on products are colony forming units and may not reflect individual microorganisms. Testing was performed in compliance with U FDA good manufacturing practice (GMP) regulations 21 C.F.R. Parts 210, 211, and 820. The procedure involved the following:

Positive Controls/Monitors: Bacillus atrophaeus

Extract Fluid: Peptone Tween

Extract Fluid Volume: ˜300 mL

Extract Method: Orbital Shaking

Plating Method: Membrane Filtration

Agar Medium: Tryptic Soy Agar
Potato Dextrose Agar

Recovery Efficiency: Exhaustive Rinse Method

Aerobic Bacteria: Plates were incubated 3-7 days at 30-35° C., then enumerated

Fungal: Plates were incubated 3-7 days at 20-25° C., then enumerated

Results

The results are reported as colony forming units (CFU) per test article. “UTD” occurs due to zero count on the first rinse.

TABLE 8

| | | | | Total | |
|---|---|---|---|---|---|
| | | | | Bioburden | Total |
| Unit | Weight | | | (CFU/test | Bioburden |
| Number | (g) | Aerobic | Fungal | article) | (CFU/g) |
| 1 | 3.7 | <3 | <3 | <6.1 | <1.7 |
| 2 | 3.6 | <3 | <3 | <6.0 | <1.7 |
| 3 | 3.8 | <3 | <3 | <6.2 | <1.6 |
| 4 | 3.6 | <3 | <3 | <6.0 | <1.7 |
| 5 | 3.6 | <3 | <3 | <6.0 | <1.7 |
| Recovery | UTD* | | | | |
| Efficiency | | | | | |

< = No Organisms Detected
UTD = Unable to Determine

### Example 5: Summary of Various Testing Results

In this example, bacterial filtration efficiency, particle filtration efficiency, viral filtration efficiency, microbial cleanliness (bioburden), resistance to penetration by synthetic blood, and flammability of clothing textiles were conducted on the test article. A summary of results is provided in the table below. The results for bacterial filtration efficiency, particle filtration efficiency, bioburden, resistance to penetration by synthetic blood, and flammability of clothing textiles are applicable to and the same for a test article having an antimicrobial silver preservative coating as described above.

TABLE 9

| Test Name | Test Code | Results | Pass/Fail |
|---|---|---|---|
| Bacterial Filtration | ASTM F2101-19 | 99.7% | Pass |
| Efficiency (BFE) | | | |
| Particle Filtration | ASTM F2299 | 99.7% | Pass |
| Efficiency (PFE) | | | |
| Viral Filltration | ASTM F2101 | 99.5% | Pass |
| Efficiency (VFE) | adaption | | |
| Bioburden | EN 14683: 2019 | <1.7 CFU/g | Pass |
| Resistance to | ASTM F1862-17 | None | Pass |
| penetration by | | | |
| synthetic blood | | | |
| Flammability of | 16 CFR 1610 | Class 1, Normal | Pass |
| clothing textiles | | Flammability | |

The standards in the table include the version in effect at the filing of present application.

### Example 6: Coating Trials

In this example, coating trials were conducted on the substrate layer of the test article. Each trial subjected the substrate layer of the test article to a different coating. Results were recorded, including comprehensive information of challenges faced and potential modes of failure.

Trial A

In this trial, one batch of substrate layer paper samples was coated with 0.5 gsm silver loading and a coat weight of 11.26 gsm. A second batch of substrate layer paper samples was coated with 1.04 gsm silver loading and a coat weight of 10.85 gsm. A range of 250-300° F. for dryer sections was identified as the limit to obtain the desired amount of silver loading on the paper. Variation in the slot die angle was observed to improve the spread of solution across the paper. Effects of gravity and slot angles may cause differential pressure across the edges of the paper.

Trial B

In this trial, the substrate layer paper samples were coated with a coat weight of 17.6 gsm and a silver weight of 0.52 gsm. A 30% solids solution was observed to be coated. Single sided versus double sided coating was validated using high viscous 30% solids solution (0.5% guar gum). The coat weight and silver loading was observed to increase to 2×from single to double side coated paper. The % silver fixation did not increase by the same extent. Discoloration of finished rolls were observed driven by ultraviolet light exposure. Increased % silver fixation from 22% to 33%, comparing the discolored and non-discolored regions, was observed.

Trial C

In this trial, the substrate layer paper samples were coated with a coat weight of 13.304 gsm and a silver loading of 0.675 gsm. Successful process validation of coating with 40% solids solution (1:10 Ag to Dextrose) was observed. Use of guar gum was observed to control the coat weight by reducing the saturation of paper, by increased soak/wicking time, as compared to the non-guar gum coating solution. The coat weight was observed to decrease from 25.0 gsm to an average of 13.0 gsm, with variations in concentrations of guar gum added (e.g. 0.35% & 0.3% for the required viscosity of 500 cPs). Coating line speed may be sped up from 15 fpm to 35 fpm with similar appearance but different coating aesthetics. Higher web speed was observed to deposit less volume of solution, with less “dwell time” in the dryer, leading to lower Ag loading & lower % silver fixation.

Trial D

In this trial, the substrate layer paper samples were subjected to an ultraviolet curing step. The solution was heated from 15° C. to 30° C., resulting in observed high shear and increased solubility of chemicals. A difficulty in complete mixing of the coating solution due to higher solids (40%) was observed. Change in gravure cell engraving leading to an increase or decrease in coat weight/add-on was also observed. The dryer temperature was increased. Higher dryer temperature leading to increased silver reduction and higher silver fixation was further observed. For example, increasing the dryer temperature from 250° F. to 275° F. increased the silver fixation from 54% to 74%.

The coated roll was treated with ultraviolet treating. Ultraviolet curing using a 40 inch ultraviolet curing line was observed to drive the silver fixation towards to completion (thereby enhancing the samples' resistance to environmental factors such as aging or silver release). For example, the silver retention was observed to increase to greater than 90% retention for all paper samples. No extreme changes in material or surface properties were observed; however, the paper samples contracted in width by a 1% decrease. Color gradients were observed due to non-uniform ultraviolet exposure from imbalanced orientation of the ultraviolet bulb. It was further observed that optimization of the process parameters of the curing line may ensure reliable and reproducible finished product for future trials.

Aging studies were conducted on the ultraviolet material. Accelerated aging on the ultraviolet cured samples were performed, with color gradients (i.e. non-uniform intensity). The regions with high ultraviolet exposure were observed to show greater resistance to aging (i.e. no change in appearance/darkening). The regions less exposed to ultraviolet light were observed to succumb to moisture and heat, leading to non-uniform discoloration of the coated paper samples.

Additional embodiments include a variety of medical and consumer applications for a substrate with a metal particles bonded to the fibers thereof, which include, but are not limited to 1) respirators (e.g. N95 respirators), 2) examination table products, 3) disposable articles, such as gowns, drapes, capes, privacy curtains, doors, and dental bibs, 4) disposable bedding articles and related supplies, 5) wound dressing, 6) high-touch products, such as secondary packaging, including paper grocery and delivery bags, paper and cardboard packaging, mailers, mail pouches, and the like; and 7) adhesive backed articles or paper configured to be adhered to surfaces (e.g. stickers, etc.).

In one embodiment, the article 100 may be a filtering facemask N95 respirator (FFM). The metallic cellulosic material may be incorporated into the FFM. The FFM may consist of multi-ply nonwoven material and may have the metallic cellulosic material added in a similar fashion to the face covering article described above.

In one embodiment, the article 100 is a disposable product for use on medical examination tables. Disposable products for use on examination tables are typically 2-ply or 3-ply tissue papers, which are cut to 40″ by 48″ or other dimensions for medical exam table paper or sold in roll form for fitting on the tables. The medical exam table paper may have a basis weight between 50 to 75 gsm, depending upon the number of plies, which are held together by thermal or chemical means. The texture of the medical exam table paper may be smooth, creped, or air-laid, and can be made from virgin or recycled wood pulp or fluff pulp. The metallic particles may be embedded in or deposited on the cellulosic or absorbent material during paper formation. Thus, the examination paper itself may have metal particles or inhibit or prevent progression of pathogens.

In one embodiment, the article is a disposable medical article. The disposal medical articles as descried herein may include a multilayered substrate composed of tissue paper, spun bond polypropylene film, and tissue paper. The polymeric barrier disposed along inside of the products may protect against fluid penetration to the skin of the user. To add metallic nanoparticles to this material, the tissue paper layers may need treatment either on or off the paper machine following methods described in PCT Publication No. WO2017124057. During the conversion process, the polymeric barrier layer would then be added through with a lamination process. At least one of the two tissue layers may have the metallic silver particles added or metallic copper particles added. The tissue paper may range from 20 to 25 gsm and may be made of recycled or virgin wood pulp. The tissue paper may be creped, smooth, or air-laid in texture. Following assembly of the three-layered material, the material may then be embossed, and cut to size and glued or otherwise attached to create disposable antimicrobial medical apparel such as patient capes, robes, and gowns, and scrub shirts and pants.

In one embodiment, the article 100 may be a disposable bedding article. Disposal bedding articles may include antiviral sheets, antimicrobial sheets, blankets, pillowcases, curtains, and other non-apparel uses. These products have differing product specifications than medical apparel. Sheeting products may be fitted or flat. Fitted sheets contain elastomeric border to aid in securing the sheet to the mattress. Disposable sheets need to be more durable than apparel, thus are typically made from a mix of rayon, cotton, air-laid cellulose, and polyester textile materials. To add antimicrobial and/or antiviral particles to these sheets, the rayon fibers and/or yarns may go through a coating process to add the metallic ion precursors and follow the synthesis methods described in the PCT Publication No. WO2017124057. Following the synthesis of metal nanoparticles on the rayon fibers, the nano-metal rayon fibers may be mixed with the polyester fibers into a knit or woven textile. Bedding articles, such as sheets, may have a base layer of polypropylene as an impenetrable layer to prevent the spread of fluids, which in medical settings may contain infectious agents. Having a top layer of an antimicrobial and/or antiviral tissue would still provide an absorbency and help retain the infectious agents in contact with the metal particle biocide.

In one embodiment, the article 100 may be a wound care product. The wound care product may include bandages and wound dressing and the like. The wound care product may include at least one substrate layer that includes metal nanoparticles. The wound care product may further include a protection layer having a substrate and metal particles in the substrate. The metal nanoparticles may have a size that ranges from 1 to about 200 nanometers in at least one dimension, wherein the metal particles are configured to inhibit or prevent pathogen growth. The metal particles include at least one of: silver, gold, platinum, palladium, aluminum, iron, zinc, copper, cobalt, nickel, manganese, molybdenum, cadmium, iridium, and a mixture thereof. The protection layer may be a paper, a textile material, a non-woven material, or a laminate thereof. The protection layer is antimicrobial and antiviral. One or both of the substrate layers and the protection layer may be absorbent. The substrate layer may be formed according to the processes and method described in PCT Publication No. WO2017124057.

In one embodiment, the article 100 may be a high-touch product or packaging. High-touch products, such as secondary packaging, including paper grocery bags, paper and cardboard packaging may be formed. Fomite surfaces can be formed on various types of packaging, grocery bags, envelopes, and cardboard boxes if the various surfaces are exposed to the infectious agent. This infectious agent can be reduced or eliminated if the packaging surface is embedded with silver particles, such as nano-silver, copper particles, such as nano-copper, etc. This process can be applied to the bulk of cellulosic materials used to produce all types of secondary packaging, including kraft pulp, recycled (recovered) pulp and paper, molded fiber (pulp), mechanical pulp, sulfite pulp, etc. Additionally, the metal precursors can be added as a surface coating to base papers, such as grocery sack paper, office paper, corrugated cardboard, other cardstock paper, mailers, mail packaging, etc., through a finishing coating process.

The article may include a protection layer having a substrate and metal particles in the substrate, the metal particles having a size that ranges from 1 to about 200 nanometers in at least one dimension, wherein the metal particles are configured to inhibit or prevent pathogen growth. The metal particles may include at least one of: silver, gold, platinum, palladium, aluminum, iron, zinc, copper, cobalt, nickel, manganese, molybdenum, cadmium, iridium, and a mixture thereof. The metal particles may further include silver or copper. The protection layer may be a paper, a textile material, a non-woven material, or a laminate thereof. The protection layer may be antimicrobial and antiviral. The packing article may be a mailer, a bag, an envelope, or a cardboard box.

In one embodiment, the article 100 may be an adhesive. Adhesive articles may include 1) a cellulosic substrate having metal particles embedded therein or components of the cellulosic substrates, such as fibers, and 2) adhesive layer to aid in adhering the article to a surface. Again, the cellulosic substrates may be formed in accordance with the method as described in PCT Publication No. WO2017124057, the entire disclosure of which is incorporated by reference in to the present disclosure. A cover layer may be applied to protect the adhesive until use. In use, the cover layer is removed, and article is placed on its intended surface. The adhesive adheres the substrate to the intended surface.

The adhesive article may include a protection layer having a substrate and metal particles in the substrate, the metal particles having a size that ranges from 1 to about 200 nanometers in at least one dimension, wherein the metal particles are configured to inhibit or prevent pathogen growth. The adhesive article may further include an adhesive disposed along one side of the protection layer. The adhesive article may further include an optional cover layer that is directly adjacent to and faces the adhesive, the optional cover layer configured to be removed so as to expose the adhesive for placement on a surface. The metal particles may include at least one of: silver, gold, platinum, palladium, aluminum, iron, zinc, copper, cobalt, nickel, manganese, molybdenum, cadmium, iridium, and a mixture thereof. The metal particles may include silver or copper. The protection layer may be paper, a textile material, a non-woven material, or a laminate thereof. The protection layer may be antimicrobial and antiviral.

Any suitable process can be used to manufacture the articles described herein. In several instances, the metal particles are added during a phase of manufacturing the substrate, such during paper forming, or via coating of roll goods, both of which are described in PCT Publication No. WO2017124057. However, the articles described in here may be manufactured using any particular means for applying metal nanoparticles thereof, including via spray mechanism or some other means. In other words, the infection inhibiting components, e.g. silver or copper, may be applied at any stage of manufacturing the articles as described herein.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1-60. (canceled)

61. A face covering article, comprising:
an inner layer configured for placement adjacent to a wearer's skin when the face covering article is applied to the face of the wearer, the inner layer including thermoplastic fibers, and an outer region that defines a perimeter of the inner layer;
a middle layer adjacent to the inner layer, the middle layer including thermoplastic fibers and an outer region that defines a perimeter of the middle layer;
an outer protection layer adjacent to the middle layer and opposite the inner layer, the outer protection layer having a blend of cellulosic fibers and thermoplastic fibers, and metal particles included therein, the metal particles being configured to inhibit or prevent pathogen growth, and an outer region that defines a perimeter of the outer protection layer, wherein at least a portion of the outer region of the inner layer and the outer region of the outer protection layer are bonded together; and
an attachment member configured to attach the face covering article to the wearer.

62. The face covering article of claim 61, wherein the metal particles are formed at least on an outer surface of the outer protection layer and have a size that ranges from 1 to about 200 nanometers in at least one dimension.

63. The face covering article of claim 61, wherein the outer protection layer includes up to about 1.5 gsm of metal particles.

64. The face covering article of claim 61, wherein the metal particles include at least one of: silver, gold, platinum, palladium, aluminum, iron, zinc, copper, cobalt, nickel, manganese, molybdenum, cadmium, iridium, and a mixture thereof.

65. The face covering article of claim 61, wherein the outer protection layer includes between 0.05 gsm to about 1.5 gsm of silver particles.

66. The face covering article of claim 65, wherein the outer protection layer includes between 0.5% and 5.0% by weight of silver per gram of the outer protection layer.

67. The face covering article of claim 61, wherein the outer protection layer is nonwoven material that includes staple cellulosic fibers and thermoplastic fibers.

68. The face covering article of claim 61, wherein the outer protection layer is antimicrobial and antiviral.

69. The face covering article of claim 61, wherein the inner layer, the middle layer, and the outer protection layer are ultrasonically welded together at their respective outer regions and one or more of the inner layer, the middle layer, and the outer protection layer has one or more pleats.

70. The face covering article of claim 61, wherein the outer protection layer has a basis weight between about 16.0 gsm and about 45.0 gsm, between 0.05 gsm to about 1.5 gsm of metal particles having a size that ranges from 1 to about 200 nanometers in at least one dimension, an antiviral log reduction of at least 2.0, an average filtration efficiency of at least 99.71%, and a bacterial filtration efficiency of at least 99.37%.

71. The face covering article of claim 61, wherein the inner layer and the middle layer are laminates of spunbond and meltblown substrates.

72. The face covering article of claim 61, wherein the outer protection layer includes an antimicrobial silver preservative coating having between 0.2% and 1.0% by weight of silver per gram of the outer protection layer.

73. A method of manufacturing a protection layer, comprising:
applying a corona discharge treatment to a substrate having a blend of cellulosic and thermoplastic fibers;
applying an aqueous solution to the substrate, the aqueous solution having a metal precursor and reducing agent; and
applying thermal energy to the aqueous solution, thereby giving rise to metal particles on the surface of the substrate, wherein the metal particles are configured to inhibit or prevent pathogen growth.

74. The method of claim 73, wherein the substrate has a basis weight between about 16.0 gsm and about 45.0 gsm, wherein the applying thermal energy step gives rise the metal particles loaded on the substrate between 0.05 gsm to about 1.5 gsm of metal particles loaded onto the substrate, wherein the metal particles have a size that ranges from 1 to about 200 nanometers in at least one dimension, an antiviral log reduction of at least 2.0, an average filtration efficiency of at least 99.71%, and a bacterial filtration efficiency of at least 99.37%.

75. The method of claim 73, further comprising bonding the protection layer to a middle layer having a basis weight between about 20.0 gsm and about 80.0 gsm, and an inner layer having a basis weight between about 20.0 gsm and about 80.0 gsm, wherein the protection layer is a nonwoven material that includes staple cellulosic fibers and staple thermoplastic fibers, and the inner layer and the middle layer are nonwoven substrates including thermoplastic fibers.
